(19) United States
(12) Patent Application Publication
(10) Pub. No.: US 2021/0017554 A1
(43) Pub. Date: Jan. 21, 2021

(54) Enzymatic Method for Selective Catalytic Preparation of 4-Octyl Itaconate

(71) Applicants: Shanghai Xiaoran Cosmetics Co., Ltd., Shanghai (CN); Xiamen Shuangbei Biological Technology Co., Ltd., Xiamen (CN); Beijing University of Chemical Technology, Beijing (CN)

(72) Inventors: Li DENG, Beijing (CN); Changsheng LIU, Beijing (CN); Fang WANG, Beijing (CN)

(21) Appl. No.: 17/035,849

(22) Filed: Sep. 29, 2020

(30) Foreign Application Priority Data
Mar. 11, 2020 (CN) 202010165175.3

(51) Int. Cl. C12P 7/64

(52) U.S. Cl. CPC C12P 7/6454

(57) ABSTRACT

An enzymatic method for selective catalytic preparation of 4-octyl itaconate is provided, which belongs to the fields of biochemical engineering and enzymatic catalysis. The method uses a lipase or an enzyme preparation with a catalytic triplet structure (Ser-His-Asp or Ser-His-Gly) as a catalyst, itaconic acid and n-octanol or n-octanol-derived ester as raw materials to selectively catalyze the synthesis of 4-octyl itaconate in a solvent system or a solvent-free system. Compared with chemical synthesis routes of 4-octyl itaconate, the present method has the advantages of green, safety, and environmental protection. The obtained 4-octyl itaconate is one of the new small molecule compounds with anti-inflammatory and anti-viral properties.

### CROSS-REFERENCE TO RELATED APPLICATIONS

The application claims the priority of the Chinese patent application filed on Mar. 11, 2020, with the application number of CN202010165175.3 and the invention title of “ENZYMATIC METHOD FOR SELECTIVE CATALYTIC PREPARATION OF 4-OCTYL ITACONATE”, the entire contents of which are incorporated herein by reference.

### FIELD OF TECHNOLOGY

The present invention belongs to the fields of biochemical engineering and enzymatic catalysis. In particular, it relates to an enzymatic method for selective catalytic preparation of 4-octyl itaconate.

### BACKGROUND

Itaconic acid is a dicarboxylic acid containing a C═C unsaturated double bond, which has important applications in the fields of chemical engineering, polymer materials synthesis, and medicine. In recent years, as a small molecule anti-inflammatory drug, itaconic acid has attracted much attention in treating chronic inflammation, reducing Zika virus infection, and regulating metabolic pathways in the body (Hooftman, A., & O'Neill, L. A. (2019). The immunomodulatory potential of the metabolite itaconate. Trends in immunology, 40(8), 687-698). Itaconic acid can be alkylated with cysteine residues of key proteins in the metabolic pathway through Michael addition reaction, and thus play a regulatory role. For example, the alkylation reaction with the Cys 151 of KEAP1 protein activates the Nrf2 pathway, further exerting an anti-inflammatory effect (Mills, E. L., Ryan, D. G., Prag, H. A., Dikovskaya, D., Menon, D., Zaslona, Z., . . . & Szpyt, J. (2018). Itaconate is an anti-inflammatory metabolite that activates Nrf2 via alkylation of KEAP1. Nature, 556(7699), 113.). How to realize the efficient intracellular transport of itaconic acid is the key to exert the immune regulation function of itaconic acid and its derivatives. Itaconic acid, as a small molecule dicarboxylic acid, is very hydrophilic and difficult to cross the phospholipid bilayer of cells. In 2018, Mills et al. reported that 4-octyl itaconate (4-OI) could regulate immune function and exerting anti-inflammatory effects by activating the KEAP1-Nrf2 pathway (Mills, & Szpyt, (2018). Nature, 556(7699), 113.). 4-OI has a higher hydrophobicity than that of itaconic acid, which can achieve an intracellular delivery effectively. Besides, 4-OI still maintain a free α, β-unsaturated carboxylic acid, making it to mimic the endogenous itaconate better in electrophilicity. Since 2020, SARS-Cov2 has swept the world, causing a large number of infections and deaths, and seriously threatening the normal operation of the world economy. SARS-Cov2 can cause respiratory failure and death by infecting people and triggering a “cytokine storm”. The 4-octyl itaconate has a good performance in anti-inflammation (Mills, & Szpyt, (2018). Nature, 556(7699), 113.) and anti-viral (Hooftman & O'Neill. Trends in immunology. 2019, 40 (8), 687-698), such as Zika virus. Meanwhile, other similar derivatives of itaconic acid have been found to have inhibitory effects in influenza and other viral infections (Sethy, B., Hsieh, C. F., Lin, T. J., Hu, P. Y, Chen, Y L., Lin, C. Y, . . . & Hsieh, P. W. (2019). Design, synthesis, and biological evaluation of itaconic acid derivatives as potential anti-influenza agents. Journal of medicinal chemistry, 62(5), 2390-2403.). Therefore, 4-octyl itaconate can realize the effective in vivo delivery of itaconic acid, and is a potential drug for the treatment of SARS-Cov2 (Olagnier, D. P., Farahani, E., Thyrsted, J., Cadanet, J. B., Herengt, A., Idorn, M., . . . & Schilling, M. (2020). Identification of SARS-CoV2-mediated suppression of NRF2 signaling reveals a potent antiviral and anti-inflammatory activity of 4-octyl-itaconate and dimethyl fumarate.). On one hand, it may inhibit the replication of SARS-Cov2 virus; on the other hand, it can effectively reduce the inflammatory.

At present, the synthesis of 4-octyl itaconate reported is only through the ring-opening reaction of itaconic acid anhydride with n-octanol and the esterification of itaconic acid with n-octanol under acid-catalyzed conditions. Mills et al. reported the addition reaction of itaconic acid anhydride with n-octanol at room temperature, but the yield of 4-OI was only 33% (Mills, & Szpyt, (2018). Nature, 556 (7699), 113.). Gargallo et al. reported the direct esterification of itaconic acid and n-octanol under acidic conditions to prepare 4-octyl itaconate, but the yield of 4-OI prepared by this method was only 35% (Gargallo, L., Radid, D., & León, A. (1985). Polymer conformation and viscometric behaviour, 3. Synthesis, characterization and conformational studies in poly (mono-n-octyl itaconate). Die Makromolekulare Chemie: Macromolecular Chemistry and Physics, 186(6), 1289-1296.) At present, the patent reports on itaconic acid monoester concentrated more on monobutyl itaconate. Patent (CN102079702A) uses p-toluenesulfonic acid, sodium acetate or sodium bisulfate as a catalyst to prepare a mixture of dibutyl itaconate and monobutyl itaconate by controlling the reaction molar ratio. Through the optimization of various conditions in preparation and separation process, a higher purity of monobutyl itaconate was obtained with the overall yield of 60%-75%. The patent protects a chemical catalytic method that achieves high monobutyl ester conversion rate by controlling the reaction process, which has poor monoester regio-specificity and low yield, as well as the regio-specificity towards C4. Patent (CN103360251A) uses ZSM-5 zeolite as a catalyst to synthesize monobutyl itaconate, and the yield is greater than 80% with the selectivity over 90% under optimal conditions.

It should be noted that Mills et al. particularly emphasized the regio-specificity of monoesters, and 4-OI was the most effective itaconic acid derivatives that activated the KEAP1-Nrf2-ARE pathway (Mills, & Szpyt, (2018). Nature, 556(7699), 113.). Activation of ARE (antioxidant response element) can regulate the expression of antioxidant proteins, phase II detoxification enzymes, molecular chaperone genes and anti-inflammatory factor genes. Thus, it can enhance the tissue antioxidant capacity, protect tissues from poison damage, further reaching anti-tumor, anti-inflammatory function.

### SUMMARY

The present disclosure provides an enzymatic method for selective catalytic preparation of 4-octyl itaconate, which uses a lipase as a catalyst for the first time, and uses an itaconic acid and an n-octanol or its derivatives as substrates to selectively synthesize 4-octyl itaconate in a solvent system or a solvent-free system. The yield of 4-octyl itaconate is higher (93% in a solvent-free system, 98% in a solvent system). The selectivity of the obtained mono-octyl ester is near 100% (4-octyl itaconate). Enzymes are easily separated from the reaction solution, and the reaction process is environmental-friendly.

The present disclosure provides an enzymatic method for selective catalytic preparation of 4-octyl itaconate, comprising the following steps: using an itaconic acid and an n-octanol or an n-octanol-derived ester as raw materials to perform an esterification, and using a lipase to catalyze the esterification, wherein the molar ratio of the itaconic acid to the n-octanol or the n-octanol-derived ester is 1: (2-60); after the esterification is completed, performing extraction, rotary evaporation, and thermal separation to obtain 4-octyl itaconate.

Preferably, the molar ratio of the itaconic acid to the n-octanol or the n-octanol-derived ester is 1: (2-40).

More preferably, the molar ratio of the itaconic acid to the n-octanol or the n-octanol-derived ester is 1: (5-30).

Preferably, temperature of the esterification is 5-95° C., and reaction time is 4-120 hours.

Preferably, temperature of the esterification is 20-90° C., and reaction time of the esterification is 4-60 hours.

More Preferably, temperature of the esterification is 30-70° C., and reaction time is 12-48 hours.

Preferably, the lipase is derived from animals, plants and microorganisms.

More preferably, the lipase includes, but is not limited to, one of Novozym 435, Lipozyme RM IM, Lipozyme TL IM, Yarrowia lipolytica lipase, porcine pancreas lipase, Rhizopus lipase and Carica papaya lipase.

More preferably, the lipase is Novozym 435.

Preferably, the amount of the lipase is 1%-200% of the mass of the itaconic acid.

More preferably, the amount of the lipase is 10%-100% of the itaconic acid, based on mass.

More preferably, the amount of the lipase is 30-60% of the itaconic acid, based on mass.

More preferably, the amount of the lipase is 50% of the mass of the itaconic acid.

Preferably, the n-octanol-derived ester includes but is not limited to one of octyl formate or octyl acetate.

Preferably, the esterification process is carried out in a solvent system or a solvent-free system.

Whether the reaction system for the esterification of the present invention adopts a solvent system or a solvent-free system, the lipase shows a high monoester catalytic selectivity and regio-specificity.

Preferably, the esterification is carried out in a solvent system.

Preferably, the amount of the solvent is 0.2-10 times of the n-octanol or n-octanol-derived ester, based on volume.

More preferably, the amount of the solvent is 1 time of the volume of the n-octanol or n-octanol-derived ester.

Preferably, the solvent is an organic solvent, and the organic solvent includes, but is not limited to, one of chloroform, toluene, n-hexane, n-heptane, acetone, butanone, benzene, cyclohexane, and isooctane.

Preferably, the esterification process is conducted under a normal pressure condition or under a reduced pressure condition.

The normal pressure condition is 1.013×10⁵ pa; the reduced pressure condition is 800-1200 pa.

Preferably, during the esterification, the stirring speed is 50-800 rpm.

Preferably, the extraction step is as follows: adding an aqueous phase solution into the product system after the esterification in a volume ratio of 1:(0.2-20), mixing, and standing for 6-12 hours. Then, removing the aqueous phase and collecting the organic phase.

More preferably, the aqueous phase solution is saturated saline solution, that is, the saline solution is saturated by adding excess salts in water.

Preferably, the rotary evaporation step is as follows: the organic phase is subjected to rotary evaporation for 8-15 minutes at 60-80° C., 80-120 rpm, to obtain a crude product.

Preferably, the thermal separation step is as follows: the crude product after the extraction and rotary evaporation steps was carried out the thermal separation under the condition of a vacuum degree of 0.5 pa-100 pa and a heating temperature of 20° C.-100° C.

Preferably, the thermal separation step is as follows: the crude product after the extraction and rotary evaporation steps was carried out the thermal separation under the condition of a vacuum degree of 1 pa-10 pa and a heating temperature of 30° C.-50° C.

More preferably, the thermal separation condition is: the vacuum degree is 1 pa, the heating temperature is 30° C. Under this condition, a better separation of 4-octyl itaconate and n-octanol or n-octanol-derived esters can be achieved.

Preferably, the thermal separation is one of reduced pressure distillation, rotary evaporation, or short-path distillation. The separated n-octanol or n-octanol-derived ester is recyclable.

The enzymatic method for selective catalytic preparation of 4-octyl itaconate is carried out in a bioreactor.

Preferably, the reactor includes, but is not limited to, one of metal bath reactor, shaking incubator, conventional stirred reactor, vacuum reaction system, packed bed reaction system, and rotating packed bed reaction system.

Preferably, the amount of the lipase is 30-60%, the molar ratio of the itaconic acid to n-octanol or n-octanol-derived ester is 1: (5-30) (in a solvent system; the amount of the solvent is 1 time of the volume of n-octanol or n-octanol-derived ester); the esterification temperature is 30-70° C.; the stirring speed is 200 rpm, and the esterification time is 12-48 hours. The process has the advantages of high monoester conversion, high monoester selectivity, strong regio-specificity, mild conditions, simple separation, high catalytic efficiency, short reaction time, etc.

Preferably, the amount of the lipase is 50%, the molar ratio of the itaconic acid to n-octanol or n-octanol-derived ester is 1:20 (in a solvent system; the molar ratio is 1:10. And the amount of the solvent is 1 time of the volume of n-octanol or n-octanol-derived ester); the esterification temperature is 50° C.; the stirring speed is 200 rpm, and the esterification time is 36 hours (in a solvent system, the esterification time is 24 hours).

Preferably, in the esterification, filtering the reaction solution through a Buchner funnel to remove enzymes, then pouring it into a separatory funnel, adding saturated saline in the volume ratio of 1:(0.2-20), mixing, and standing for 6-12 hours, and separating the lower aqueous phase to remove itaconic acid; collecting the organic phase, removing trace moisture and solvent by rotary evaporation (70° C., 100 rpm, 10 minutes), the heavy phase is used in the subsequent thermal separation process, and the separated solvent is recyclable.

### Beneficial Effects

The present invention provides an enzymatic method for selective catalytic preparation of 4-octyl itaconate, which has the advantages of mild reaction conditions, simple separation, strong selectivity, high catalytic efficiency, short reaction time and long cycle life.

(1) The catalyst of the present invention utilizes the steric effect of specific catalytic active pocket and high regio-specificity of the lipase, which helps to improve the selectivity of the mono-esterification process; and the carboxyl group at the C1 position of itaconic acid is close to the C═C double bond at the C2 position (see FIG. 2), and has higher steric hindrance into the enzyme active pocket. Therefore, the esterification activity of carboxyl group at the C4 position of itaconic acid is greater than that at the C1 position, thereby achieving a higher yield of 4-octyl itaconate;

(2) The present invention utilizes the microphase distribution of the reaction substrate under the lipase-catalyzed microenvironment. After the mono-esterification of itaconic acid, driven by the hydrophobic force, the formed itaconic acid monoester will leave the catalytic site of the enzyme and spread into the surrounding hydrophobic environment instantly, then, a large amount of itaconic acid monoester is accumulated;

(3) The catalyst of the present invention is a lipase catalyst, which has the advantages of easy to obtain, easy to separate, mild reaction conditions requirement, environmental-friendly process, and high batch life.

(4) The present invention utilizes thermal separation method to achieve the separation of excess n-octanol or n-octanol-derived ester from the target product through the difference of saturated vapor pressure of different components. The process is simple to operate, the product purity is high, and it is easy to industrialize.

(5) The present invention achieves a high conversion rate of itaconic acid, and the conversion rate of which is as high as 99%. The yield of 4-octyl itaconate is as high as 98.5%, and at the same time, the preparation method of the present invention achieves a near 100% selectivity of 4-octyl itaconate (see FIG. 4 to FIG. 8).

### BRIEF DESCRIPTION OF THE DRAWINGS

FIG. 1 shows an esterification process of itaconic acid and n-octanol or n-octanol-derived ester in the presence of a conventional catalyst;

FIG. 2 shows an esterification process of itaconic acid and n-octanol or n-octanol-derived ester in the presence of a lipase;

FIG. 3 is a gas chromatogram of the content of components in the reaction solution in a solvent system condition in embodiment 1 of the present invention;

FIG. 4 is a gas chromatogram of the content of each component in the separated product in embodiment 1 of the present invention;

FIG. 5 shows a nuclear magnetic structure identification-H spectrum-600M of the product and the standard product in experimental example 1 of the present invention;

FIG. 6 shows a nuclear magnetic structure identification-C spectrum-150M of the product and the standard product in experimental example 1 of the present invention;

FIG. 7 shows a two-dimensional nuclear magnetic structure identification (HIBC)-600M of the product in experimental example 1 of the present invention;

FIG. 8 shows a two-dimensional nuclear magnetic structure identification HMBC-600M and the local enlargement of the product in experimental example 1 of the present invention.

### DESCRIPTION OF THE EMBODIMENTS

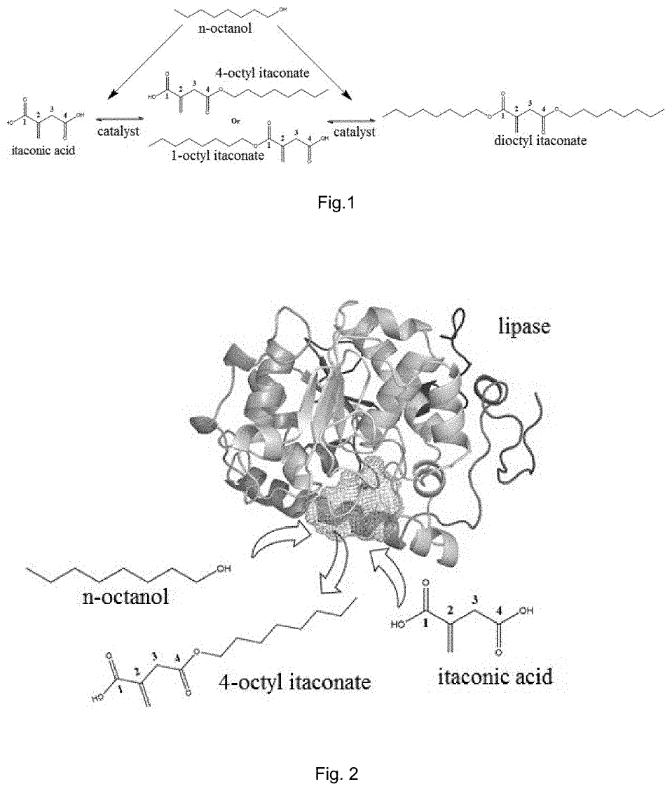

The synthesis of itaconic acid monoester either uses high-cost itaconic acid anhydride as a substrate, or forms more by-products, and the conversion rate of the monoester is not high, with a higher subsequent separation cost. The main difficulties of the preparation process of 4-octyl itaconate using itaconic acid and n-octanol as substrates lie in the following two points: First, in the cascade reaction of itaconic acid to form monoesters and diesters (see FIG. 1), a higher conversion rate of monoester is required, and the formation of the by-product diester should be reduced to lower the subsequent separation cost; Second, a higher regio-specificity is required in the generated monoester, that is, 4-octyl itaconate is formed (See FIG. 1) instead of the by-product, 1-OI. Thus, a highly selective synthetic route of 4-OI with important medical and material application prospects is in urgent need of development.

An enzymatic method for selective catalytic preparation of 4-octyl itaconate is provided in the present invention, comprising the following steps: using an itaconic acid and an n-octanol or an n-octanol-derived ester as raw materials, and adding a lipase to carry out esterification, wherein the molar ratio of the itaconic acid to the n-octanol or n-octanol-derived ester is 1: (2-60); after the esterification is completed, conducting extraction, rotary evaporation, and thermal separation to obtain 4-octyl itaconate. The esterification process of the itaconic acid with the n-octanol or n-octanol-derived ester refers to FIG. 2.

Based on the hydrophilic-hydrophobic difference of itaconic acid and 4-octyl itaconate, wherein itaconic acid is more soluble in water, n-octanol or n-octanol-derived ester, 4-octyl itaconate, and organic solvents are insoluble in water. Then, the unreacted trace itaconic acid can be removed by extraction (water-organic phase); after extraction, the organic phase contains traces of water or organic solvents (such as toluene, etc.) in the reaction process of the solvent system, which can be removed by rotary evaporation; at this time, the reaction system contains only excessive n-octanol or n-octanol-derived ester and 4-octyl itaconate, and n-octanol or n-octanol-derived ester can be separated by thermal separation (under specific pressure and temperature conditions).

Temperature of the esterification is 5-95° C., and reaction time is 4-120 hours.

As an embodiment of the present invention, the molar ratio of the itaconic acid to the n-octanol or n-octanol-derived ester is 1:2, 1:4, 1:6, 1:8, 1:10, 1:12, 1:14, 1:16, 1:18, 1:20, 1:22, 1:24, 1:26, 1:28, 1:30, 1:32, 1:34, 1:36, 1:38, 1:40, 1:42, 1:44, 1:46, 1:48, 1:50, 1:52, 1:54, 1:56, 1:58, 1:60. Preferably, the molar ratio of the itaconic acid to the n-octanol or n-octanol-derived ester is 1: (2-40). More preferably, the molar ratio of the itaconic acid to the n-octanol or n-octanol-derived ester is 1: (5-30).

As an embodiment of the present invention, the reaction temperature is 5° C., 10° C., 15° C., 20° C., 25° C., 30° C., 35° C., 40° C., 45° C., 50° C., 55° C., 60° C., 65° C., 70° C., 75° C., 80° C., 85° C., 90° C., 95° C., and the reaction time can be 4 h, 10 h, 20 h, 30 h, 40 h, 50 h, 60 h, 70 h, 80 h, 90 h, 100 h, 110 h, 120 h. Preferably, the reaction condition is at 20-90° C., and the reaction time is 4-60 hours; more preferably, the reaction condition is at 30-70° C. and the reaction time is 12-48 hours.

The lipase can show a higher enzyme activity at 20-90° C.; the reaction is a cascade reaction, and a shorter reaction time will result in a lower conversion rate of itaconic acid, and a longer reaction time will lead to an increase in the content of dioctyl itaconate; the molar ratio of the substrates will affect the yield of the product, and the higher amount of the n-octanol or n-octanol-derived ester will increase the difficulty of the subsequent separation.

The lipase is derived from animals, plants or microorganisms.

In other embodiments of the present invention, a biologic enzyme preparation containing a catalytic triplet structure (Ser-His-Asp or Ser-His-Gly) can replace the lipase to catalyze the esterification reaction of the present invention. The first step in the lipase catalytic process is the formation of acylated complex. Undergoing a series of electron transfer of amino acid residues (Asp/Gly and His) in the lipase activity center, the hydroxyl oxygen of serine (Ser) is activated and binds to the carbonyl carbon in the carboxyl group of the substrate (itaconic acid), to form an enzyme-acyl complex (Acyl-enzyme). The second step is the deacylation reaction. The nucleophilic reagent (octanol) in the reaction system will attack the carbonyl carbon in lipase acyl complex to form a new ester bond. At the same time, the enzyme-acyl complex undergoes deacylation, the substrate was released, and lipase molecules can again enter into the next catalytic cycle. Thus, other enzyme preparations containing catalytic triplet structures (Ser-His-Asp or Ser-His-Gly) can also complete the above esterification reaction, such as esterases and proteases.

Preferably, the lipase includes, but is not limited to, one of Novozym 435, Lipozyme RM IM, Lipozyme TL IM, Yarrowia lipolytica lipase, porcine pancreas lipase, Rhizopus lipase and Carica papaya lipase.

Among the above seven lipases, the Novozym 435 is derived from Aspergillus niger, and immobilized in a hydrophobic macroporous resin, purchased from Novozymes; the Lipozyme RM IM is derived from Aspergillus oryzae, and immobilized in a phenolic resin, purchased from Novozymes; the Lipozyme TL IM is derived from Thermomyceslanuginosa, and immobilized in silica, purchased from Novozymes; the Yarrowia lipolytica lipase (LS-20) is derived from Yarrowia lipolytica, powder particles, purchased from Beijing Kaitai Corporation; the porcine pancreatic lipase (CAS No. 9001-62-1), powder particles, purchased from TCI Corporation; the Rhizopus lipase is derived from Rhizopus fermentation, powder; the Carica papaya lipase is derived from papaya plants, powder.

More preferably, the lipase is Novozym 435. Under the most optimal conditions, the conversion rate of itaconic acid is greater than 98% (characterized by gas chromatography), and the selectivity of 4-octyl itaconate is near 100%.

As an embodiment of the present invention, the amount of the lipase is 10%, 20%, 30%, 40%, 50%, 60%, 70%, 80%, 90%, 100%, 110%, 120%, 130%, 140%, 150%, 160%, 170%, 180%, 190%, and 200% of mass of the itaconic acid. Higher lipase amount will shorten the reaction time, but will increase the cost; too low lipase amount can not achieve a better conversion rate of 4-octyl itaconate.

Preferably, the amount of the lipase is 10%-100% of mass of the itaconic acid. Higher lipase amount may lead to the formation of by-product, dioctyl itaconate. Lipase under preferred condition, a more outstanding conversion rate of 4-octyl itaconate can be achieved.

More preferably, the amount of the lipase is 30-60% of mass of the itaconic acid.

The n-octanol-derived ester includes, but is not limited to one of octyl formate or octyl acetate.

The esterification process is carried out in a solvent system or a solvent-free system.

Whether the reaction system of the present invention adopts a solvent system or a solvent-free system, the lipase shows a high monoester selectivity and regio-specificity.

Preferably, the esterification is carried out in a solvent system. Lipase-catalyzed esterification depends on its catalytic triplet structure located in the pocket of limited active center. After the itaconic acid is catalyzed to react with n-octanol or n-octanol-derived esters, the product, 4-octyl itaconate, has an increased hydrophobicity. In a solvent system, it is more conducive for the generated 4-octyl itaconate to come off from the catalytic active center and the accumulation of 4-octyl itaconate in the solvent is achieved through the microscopic phase distribution around the enzyme.

As an embodiment of the present invention, the amount of the solvent is 0.2, 1, 2, 3, 4, 5, 6, 7, 8, 9, or 10 times of the volume of n-octanol or n-octanol-derived ester.

The solvent includes, but is not limited to, one of chloroform, toluene, n-hexane, n-heptane, acetone, butanone, benzene, cyclohexane or isooctane.

The esterification process is under a normal pressure condition or under a reduced pressure condition.

The normal pressure condition is 1.013×10⁵ pa; the reduced pressure condition is 1000 pa.

Preferably, during the esterification, the stirring speed is 50-800 rpm.

In the extraction, rotary evaporation, and thermal separation processes, a small amount of unreacted itaconic acid after the esterification can be extracted and separated by a two-phase system (aqueous phase-organic phase) to remove itaconic acid; the organic phase is collected, and used in the subsequent separation process after removing trace water and organic solvents (such as toluene) by rotary evaporation. The separated solvent can be recycled.

Preferably, the extraction step is as follows:

Adding an aqueous phase solution into the product system after the esterification in a volume ratio of 1:(0.2-20), mixing, and standing for 6-12 hours. Then removing the aqueous phase and collecting the organic phase.

More preferably, the aqueous phase solution is saturated saline.

As an embodiment of the present invention, adding saturated saline into the product system after the esterification in a volume ratio of 1:1, mixing, and standing for 12 hours. Removing the lower aqueous phase to remove itaconic acid, then, collecting the organic phase. The aqueous phase for extracting fewer unreacted itaconic acid includes, but is not limited to, deionized water, saturated saline, and aqueous phases containing various ion concentrations. The organic phase for extracting fewer unreacted itaconic acid includes itaconic acid ester, excess octanol, and/or organic solvent.

The rotary evaporation step is as follows: the organic phase is subjected to rotary evaporation for 8-15 minutes at 60-80° C., 80-120 rpm to obtain a crude product. That is, the trace amount of water is removed in the solvent-free reaction system, while the trace amount of water and solvent are removed in the solvent reaction system. The organic phase contains trace amounts of water or organic solvents (such as toluene, etc.) in the reaction process of the solvent system, which can be removed by rotary evaporation.

The thermal separation step is as follows: carrying out the thermal separation to the crude product obtained by extraction and rotary evaporation steps under the condition of a vacuum degree of 0.5 pa-100 pa and a heating temperature of 20° C.-100° C. The obtained white solid is 4-octyl itaconate.

After the solvent is subjected to rotary evaporation, the reaction system only contains excess n-octanol or n-octanol-derived ester and 4-octyl itaconate. The atmospheric boiling point of n-octanol or n-octanol-derived esters is greater than 190° C. Under conventional rotary evaporation conditions, it is difficult to remove n-octanol or n-octanol-derived esters. Besides, the polarities of 4-octyl itaconate and n-octanol are relatively close, so it is difficult to separate by silica gel column chromatography. However, the difference in boiling point of the two substances is obvious. Under certain pressure and temperature conditions, the separation of 4-octyl itaconate and n-octanol or n-octanol-derived ester can be achieved by thermal separation (under specific pressure and temperature conditions).

Preferably, the thermal separation step is as follows: carrying out the thermal separation to the crude product obtained by extraction and rotary evaporation steps under the condition of a vacuum degree of 1 pa-10 pa and a heating temperature of 30° C.-50° C.

More preferably, the thermal separation condition is: the vacuum degree is 1 pa, the heating temperature is 30° C. Under this condition, a better separation of 4-octyl itaconate and n-octanol or n-octanol-derived esters can be achieved.

The thermal separation is one of reduced pressure distillation, rotary evaporation, or short-path distillation. The separated n-octanol or n-octanol-derived ester can be recycled. The reactor and the reaction system include, but is not limited to, a metal bath reactor, a shaking incubator, a conventional stirred reactor, a normal pressure/vacuum reaction system, a packed bed reaction system, a rotating packed bed reaction system, etc.

Preferably, the amount of the lipase is 30-60%; the molar ratio of the itaconic acid to n-octanol or n-octanol-derived ester is 1: (5-30) (in a solvent system; the amount of the solvent is 1 time of the volume of n-octanol or n-octanol-derived ester); the esterification temperature is 30-70° C.; the stirring speed is 200 rpm, and the esterification time is 12-48 hours (in a solvent system, the esterification time is 24 hours). The process has the advantages of high monoester conversion rate, high monoester selectivity, strong specificity, mild conditions, simple separation, high catalytic efficiency, short reaction time, etc.

Preferably, the amount of the lipase is 50%; the molar ratio of the itaconic acid to n-octanol or n-octanol-derived ester is 1:10 (in a solvent system; the amount of the solvent is 1 time of the volume of n-octanol or n-octanol-derived ester); the esterification temperature is 50° C.; the stirring speed is 200 rpm, and the esterification time is 36 hours (in a solvent system, the esterification time is 24 hours).

The present invention applies the lipase to the octyl esterification of itaconic acid for the first time. In a solvent-free system, the yield of 4-octyl itaconate can reach 93% (98% in a solvent system), and the selectivity of 4-octyl itaconate in monoester reaches near 100% (also near 100% in a solvent system).

In the esterification, the reaction solution is filtered through a Buchner funnel to remove enzymes, then poured into a separatory funnel, and added with saturated saline, mixed and let stand for 6-12 hours, and the lower aqueous phase is separated to remove itaconic acid; organic phase is collected, and subjected to rotary evaporation (70° C., 100 rpm, 10 minutes) to remove trace moisture and solvent, the heavy phase is used in the subsequent separation process, and the separated solvent can be recycled.

The present invention will be further described below in conjunction with specific embodiments. It should be understood that the following embodiments are for better illustrating the present invention, rather than limiting the description.

### Embodiment 1: Preparation of 4-octyl Itaconate Under Normal Pressure in a Solvent System

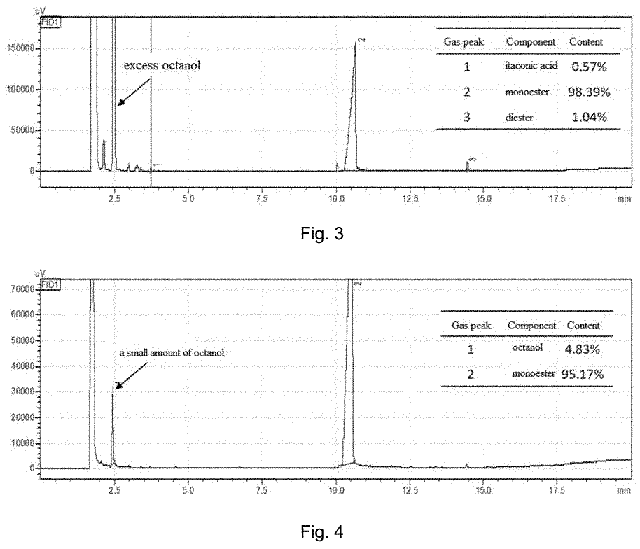

Step 1. Under normal pressure (1.013×10⁵ pa), using 1 g of itaconic acid (7.69 mmol) and 10 g (76.9 mmol) of n-octanol as raw materials, adding 0.5 g of Novozym 435 (10000 U/g) and 12 mL of solvent toluene (toluene:n-octanol=1:1, volume ratio), carrying out the esterification in a shaking incubator of 200 rpm for 24 hours at 50° C. The gas chromatography determined that the conversion rate of itaconic acid was 99%, and the yield of 4-octyl itaconate was 98% (see FIG. 3);

Step 2. After the esterification was completed, removing the lipase by filtration through a solvent filter (nylon filter membrane of 0.45 μm), pouring the reaction solution into a separatory funnel, adding a saturated saline in the volume ratio of the reaction solution to the saturated saline of 1:1 (concentration was about 35%), fully mixing and shaking the reaction mixture, and standing for 6 hours, removing the lower aqueous phase, and collecting the organic phase. The organic phase was suffered from rotary evaporation (70° C., 100 rpm, 10 minutes) to remove the trace amount of water and toluene to obtain a crude product;

Step 3. Removing the excess n-octanol of the crude product prepared in step 2 through a thermal separation method of short-path distillation, and the condition of the short-path distillation was set as follows: the temperature of an outer heating wall was 30° C., the temperature of an inner condensation was 2° C., the scraper speed was 150 rpm, the feed speed was 1 mL/min, and the vacuum degree was 1 pa. A white solid obtained after removing the excess n-octanol was 4-octyl itaconate, with a total yield of 89%, and a purity of 95% (see FIG. 4);

Step 4. Using the lipase for 16 batches of experiments, and the enzyme activity was kept above 90% of the initial enzyme activity.

### Embodiment 2: Preparation of 4-octyl Itaconate Under Reduced Pressure in a Solvent System

Step 1. Under reduced pressure (1000 pa), using 1 g of itaconic acid (7.69 mmol) and 10 g (76.9 mmol) of n-octanol were used as raw materials, and putting them into a three-necked flask, and adding 0.5 g of Novozym 435 (10000 U/g) and 12 mL of solvent toluene (toluene:n-octanol=1:1, volume ratio), controlling the water bath at 40° C., and carrying out the esterification at 200 rpm for 12 h, in a shaking incubator with a glass water segregator; The gas chromatography determined that the conversion rate of itaconic acid was 99%, and the yield of 4-octyl itaconate was 97%;

Step 2. After the esterification was completed, removing the lipase by filtration through a solvent filter (nylon filter membrane of 0.45 μm), pouring the reaction solution into a separatory funnel, adding a saturated saline in the volume ratio of the reaction solution to the saturated saline of 1:1, fully mixing and shaking the reaction mixture, and standing for 6 hours, removing the lower aqueous phase, and collecting the organic phase. Conducting rotary evaporation (70° C., 100 rpm, 10 minutes) on the organic phase to remove the trace amount of water and toluene from the supernatant organic phase to obtain a crude product;

Step 3. Removing the excess n-octanol of the crude product prepared in step 2 through a thermal separation method of short-path distillation, and the condition of the short-path distillation was set as follows: the temperature of an outer heating wall was 30° C., the temperature of an inner condensation was 2° C., the scraper speed was 150 rpm, the feed speed was 1 mL/min, and the vacuum degree was 1 pa. A white solid obtained after removing excess n-octanol was 4-octyl itaconate, with a total yield of 90% and a purity of 94%.

During the esterification of n-octanol and itaconic acid, water was continuously generated. Compared with normal pressure reaction conditions, under reduced pressure (1000 pa), water was continuously removed to push the reaction forward (i.e., the direction of esterification), thus significantly shortened the reaction time from 24 hours under normal pressure to 12 hours under reduced pressure.

### Embodiment 3: Continuously Preparation of 4-octyl itaconate in a Packed Bed Reactor in a Solvent System

Step 1. Under normal pressure (1.013×10⁵ pa), using 5 g of itaconic acid (38.45 mmol) and 50 g (384.5 mmol) of n-octanol as raw materials, adding 60 mL of solvent toluene (toluene:n-octanol=1:1, volume ratio) and mixing evenly, which can be used as a reaction substrate;

Step 2. Adding 2.5 g of Novozym 435 (10000 U/g) into a steel jacketed packed bed reactor with a length of 20 cm, an inner diameter of 1 cm, and an outer diameter of 2 cm, whose ends were filled with glass beads, pumping the reaction substrate into the packed bed reactor from bottom to top by a plunger pump with a flow rate of 0.4 mL/min (10 minutes residence time), and controlling the jacket temperature at 30° C. by a circulating water bath. The gas chromatography determined that the conversion rate of itaconic acid was 51%, and the yield of 4-octyl itaconate was 50%; when the feed was recycled for 4 times, the conversion of itaconic acid was 95%, and the yield of 4-octyl itaconate was 94%. The total residence time of the reaction solution was 40 min.

Step 3. After the esterification was completed (no enzyme removal was required), pouring the reaction solution into a separatory funnel, and adding a saturated saline in the volume ratio of the reaction solution to saturated saline of 1:1, fully mixing and shaking the reaction mixture, and standing for 6 hours, removing the lower aqueous phase, and collecting the organic phase, conducting the organic phase to rotary evaporation (70° C., 100 rpm, 10 minutes), to remove the trace amount of water and toluene to obtain a crude product; since water is insoluble in toluene, directly removing the supernatant toluene and obtaining 58 mL of toluene.

Step 4. Removing the excess n-octanol of the crude product prepared in step 3 through a thermal separation method of rotary evaporation, and the condition of rotary evaporation was set as follows: heating temperature was 120° C., condensation temperature was −5° C., rotation speed was 110 rpm, and vacuum degree was 1 pa. A white solid obtained after removing excess n-octanol was 4-octyl itaconate, with a total yield of 88%, and a purity of 91%.

Due to the mass transfer effect of the substrate material and the enzyme catalyst was slightly lower in packed bed reactors than that of direct mixing, resulting in a slight decrease in conversion rate. Moreover, as a means of thermal separation, rotary evaporation was not as efficient as short-path distillation, thus resulting in a lower purity of target product.

### Embodiment 4: Preparation of 4-octyl itaconate in a Metal Bath Reactor in a Solvent System

Step 1. Under normal pressure (1.013×10⁵ pa), using 0.1 g of itaconic acid (0.769 mmol) and 1 g (7.69 mmol) of n-octanol as raw materials and adding into a 4 mL brown vial, and adding 0.05 g of Novozym 435 (10000 U/g) and 1.2 mL of solvent toluene (toluene:n-octanol=1:1, volume ratio), carrying out the esterification in a metal bath reactor at 800 rpm for 20 hours at 50° C.; a gas chromatography determined that the conversion rate of itaconic acid was 98%, and the yield of 4-octyl itaconate was 97%;

Step 2. After the esterification was completed, removing the lipase by filtration through a solvent filter (nylon filter membrane of 0.45 μm). Pouring the reaction solution into a separatory funnel, and adding a saturated saline in the volume ratio of the reaction solution to the saturated saline of 1:1, fully mixing and shaking the reaction mixture, and standing for 6 hours, removing the lower aqueous phase, and collecting the organic phase. Conducting the organic phase to rotary evaporation (70° C., 100 rpm, 10 minutes), to remove the trace amount of water and toluene from the supernatant organic phase to obtain a crude product;

Step 3. Removing the excess n-octanol of the crude product prepared in step 2 through a thermal separation method of reduced pressure distillation, and the condition of reduced pressure distillation was set as follows: heating temperature was 150° C., temperature of circulating water condensation was 18° C., 2 zeolites, vacuum degree was 3×10² mbar, heating time was 30 minutes. A white solid obtained after removing excess n-octanol was 4-octyl itaconate, with a total yield of 80%, and a purity of 94%.

During the depressurization process, part of the product was steamed into the condenser along with n-octanol; resulting in a slight decrease in product yield.

### Embodiment 5: Preparation of 4-octyl itaconate Under Normal Pressure in a Solvent-Free System

Step 1. Under normal pressure (1.013×10⁵ pa), using 1 g of itaconic acid (7.69 mmol) and 20 g (153.8 mmol) of n-octanol as raw materials, and adding 0.5 g of Novozym 435 (10000 U/g), carrying out the esterification in a shaking incubator of 200 rpm for 36 hours at 50° C. under the condition of a solvent-free system. A gas chromatography determined that the conversion rate of itaconic acid was 98%, and the yield of 4-octyl itaconate was 93%.

In a solvent system, it was more conducive for the mono-octyl ester to remove from the catalytic site of the enzyme molecule instantly; while in a solvent-free system, the accumulation amount was slightly lower.

Step 2. After the esterification was completed, removing the lipase by filtration through a solvent filter (nylon filter membrane of 0.45 μm), pouring the reaction solution into a separatory funnel, adding a saturated saline in the volume ratio of the reaction solution to the saturated saline of 1:1, fully mixing and shaking the reaction mixture, and standing for 6 hours, removing the lower aqueous phase and collecting the organic phase. Conducting the organic phase to rotary evaporation (70° C., 100 rpm, 10 minutes) to remove the trace amount of water from the supernatant organic phase to obtain a crude product;

Step 3. Removing the excess n-octanol of the crude product prepared in step 2 through a thermal separation method of short-path distillation, and the condition of the short-path distillation was set as follows: the temperature of an outer heating wall was 30° C., the temperature of an inner condensation was 2° C., the scraper speed was 150 rpm, the feed speed was 1 mL/min, and the vacuum degree was 1 pa. A white solid obtained after removing excess n-octanol was 4-octyl itaconate, with the yield of 84%, and a purity of 90%.

In the above embodiment of the present invention, the esterification conversion rate was slightly lower under normal pressure, and the product yield might be reduced due to the emulsification of the organic phase and the aqueous phase.

### Embodiment 6: Preparation of 4-octyl itaconate Under Reduced Pressure in a Solvent-Free System

Step 1. Under reduced pressure (1000 pa), using 1 g of itaconic acid (7.69 mmol) and 20 g (153.8 mmol) of n-octanol as raw materials, and adding them into a three-necked flask, adding 0.5 g of Novozym 435 (10000 U/g), controlling the water bath at 40° C., and controlling the vacuum degree of the reaction system at 1000 Pa by a vacuum system, carrying out the esterification for 20 hours at 200 rpm in a water bath. A gas chromatography determined that the conversion rate of itaconic acid was 99%, and the yield of 4-octyl itaconate was 90%;

Step 2. After the esterification was completed, removing the lipase by filtration through a solvent filter (nylon filter membrane of 0.45 μm), pouring the reaction solution into a separatory funnel, adding a saturated saline in the volume ratio of the reaction solution to saturated saline of 1:1, fully mixing and shaking the reaction mixture, and standing for 6 hours, removing the lower aqueous phase, and collecting the organic phase. Conducting the organic phase to rotary evaporation (70° C., 100 rpm, 10 minutes) to remove the trace amount of water was removed from the supernatant organic phase to obtain a crude product;

Step 3. Removing the excess n-octanol of the crude product prepared in step 2 was through a thermal separation method of short-path distillation, and the condition of the short-path distillation was set as follows: the temperature of an outer heating wall was 30° C., the temperature of an inner condensation was 2° C., the scraper speed was 150 rpm, the feed speed was 1 mL/min, and the vacuum degree was 1 pa. A white solid obtained after removing excess n-octanol was 4-octyl itaconate, with the yield of 83%, and the purity of 88%.

The low purity of the crude product to be separated results in a slight decrease in purity of product after separation.

### Embodiment 7: Preparation of 4-octyl itaconate Using Octyl Formate as a Substrate Under Normal Pressure in a Solvent-Free System

Step 1. Under normal pressure (1.013×10⁵ pa), using 1 g of itaconic acid (7.69 mmol) and 24 g (151.7 mmol) of octyl formate as raw materials, and adding 0.5 g of Novozym 435 (10000 U/g), carrying out the esterification in a shaking incubator of 200 rpm for 30 hours at 50° C. under the condition of a solvent-free system. A gas chromatography determined that the conversion rate of itaconic acid was 96%, and the yield of 4-octyl itaconate was 94%; Step 2. After the esterification was completed, removing the lipase by filtration through a solvent filter (nylon filter membrane of 0.45 μm), pouring the reaction solution into a separatory funnel, and adding a saturated saline in the volume ratio of the reaction solution to saturated saline of 1:1.5, fully mixing and shaking the reaction mixture, and standing for 6 hours, removing the lower aqueous phase, and collecting the organic phase. Conducting the organic phase to rotary evaporation (70° C., 100 rpm, 10 minutes) to remove the trace amount of water and toluene, yielding a crude product of 4-OI;

Step 3. Removing the excess octyl formate of the crude product prepared in step 2 through a thermal separation method of short-path distillation, and the condition of the short-path distillation was set as follows: the temperature of an outer heating wall was 30° C., the temperature of an inner condensation was 2° C., the scraper speed was 150 rpm, the feed speed was 1 mL/min, and the vacuum degree was 1 pa. A white solid obtained after removing excess octyl formate was 4-octyl itaconate, with the yield of 85%, and a purity of 92%.

Compared with Embodiment 5, n-octanol derivatives, such as octyl formate was used as reaction substrates as well as n-octanol, which had little effect on the conversion rate and purity of the target product, and the yield of the target product (4-octyl itaconate) was slightly higher when n-octanol derivatives, octyl formate was used as a reaction substrate, which may be due to its less inhibitory effect on lipase activity, while the hydroxyl group (—OH) of n-octanol might affect part of the enzyme activity of the lipase.

### Experimental Example 1: Characterization of the NMR Structure of the Product

In order to characterize the structural properties of the product obtained by the preparation method of the present invention, the nuclear magnetic structures of the product prepared by the present invention and the 4-octyl itaconate standard were verified by comparison.

The experimental steps were as follows:

Step 1. Dissolving 30 mg of the high-purity product prepared in Embodiment 1 (purity ≥95%) in an appropriate amount of deuterated chloroform, putting it into a nuclear magnetic tube, and mixing evenly; preparing a nuclear magnetic sample as a reference according to the same procedure with using a 4-octyl itaconate standard (commercial standard, Ark, AK00807135, 98% purity, 30 mg);

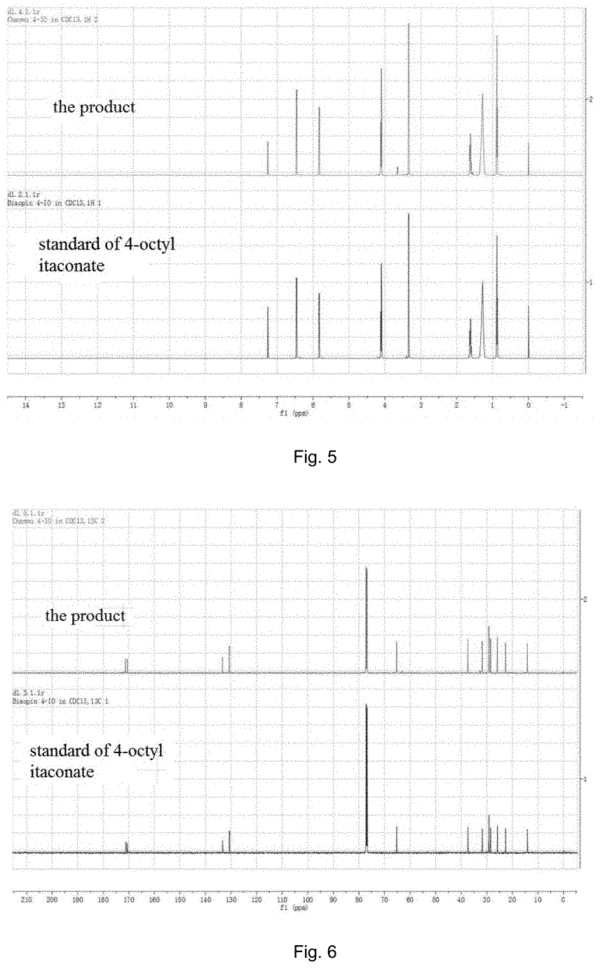

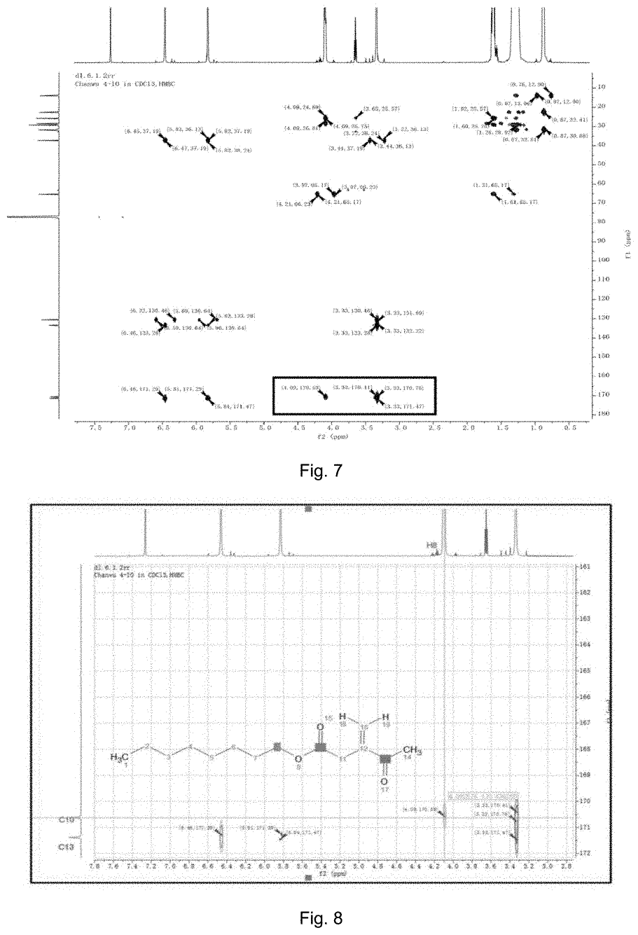

Step 2. Measuring the H spectrum and C spectrum of the product and standard, and the two-dimensional nuclear magnetic spectrum (HMBC) of the product in the Bruker600 M magnetic resonance analyzer; (see FIG. 5, FIG. 6, FIG. 7 and FIG. 8)

Step 3. The measured H spectrum and C spectrum of the product of the present invention were completely corresponding to the H spectrum and C spectrum of the standard. According to literature reports (Richard, J V, Delaite, C., Riess, G., & Schuller, A S (2016). A comparative study of the thermal properties of homologous series of crystallisable n-alkyl maleate and itaconate monoesters. Thermochimica acta, 623, 136-143), the C═C terminal hydrogen of 4-octyl itaconate and 1-octyl itaconate would have obvious deviation; and the H spectrum data of the product of the present patent was consistent with the H spectrum data of 4-octyl itaconate reported by Richard et al.; the two-dimensional nuclear magnetic spectrum (HMBC) further showed that the product of the present invention almost contain no 1-octanoic acid mono-octyl ester, and almost all of mono-octyl itaconate was 4-octyl itaconate.

### Experimental Example 2: The Effect of Different Lipases on the Yield of 4-Octyl Itaconate in a Solvent-Free System Under Normal Pressure

In order to illustrate the effect of different lipases and conventional catalysts on the conversion rate of 4-octyl itaconate, the experiments were divided into eight groups, among which:

Experimental group 1: using 0.5 g of Novozym 435 (purchased from Novozymes) as a catalyst;

Experimental group 2: using 0.5 g of Novozymes RM IM lipase (purchased from Novozymes) as a catalyst;

Experimental group 3: using 0.5 g of Novozymes TL IM lipase (purchased from Novozymes) as a catalyst;

Experimental group 4: using 0.5 g of Yarrowia lipolytica lipase (LS-20) as a catalyst;

Experimental group 5: using 0.5 g of porcine pancreatic lipase (CAS No. 9001-62-1, purchased from TCI Corporation) as a catalyst;

Experimental group 6: using 0.5 g of Rhizopus lipase, derived from rhizopus fermentation, powder;

Experimental group 7: using 0.5 g of Carica papaya lipase, derived from papaya plants, powder;

Control group: using 50 μL of concentrated sulfuric acid as a catalyst.

Experimental Method:

Step 1. Dividing the experiments into eight groups. Under normal pressure (1.013×10⁵ pa), using 1 g of itaconic acid (7.69 mmol) and 20 g (153.8 mmol) of n-octanol as raw materials, and respectively adding 0.5 g of Novozym 435, 0.5 g of Lipozyme RM IM, 0.5 g of Lipozyme TL IM, 0.5 g of Yarrowia lipolytica lipase (LS-20), 0.5 g of porcine pancreatic lipase, 0.5 g of Rhizopus lipase, 0.5 g of Carica papaya lipase, and 50 μL of concentrated sulfuric acid, which are used as catalysts. Under the condition of a solvent-free system, carrying out the esterification in a shaking incubator at 200 rpm at 50° C. for 36 hours.

Step 2. Taking out 20 μL of the esterified product from each reaction system, and adding 1.8 mL of methanol. Centrifugating the mixture at a high-speed (8000 rpm, 3 min) to remove the free enzyme protein; taking out 1.6 mL of the supernatant respectively and putting into the injection bottle, and measuring the yield of 4-octyl itaconate by gas chromatography.

Step 3. The experimental results showed that: when concentrated sulfuric acid was used as a catalyst, most of itaconic acid was converted to dioctyl itaconate (about 70%), and only a small amount of 4-octyl itaconate (about 30%) was produced. Meanwhile, lipase had the advantages of easy separation, mild reaction conditions, etc.; and all the generated mono-octyl ester was 4-octyl itaconate, but the yield of 4-octyl itaconate was different. Using Novozym 435, Lipozyme RM IM, Lipozyme TL IM, Yarrowia lipolytica lipase, porcine pancreas lipase as catalysts, Rhizopus lipase and Carica papaya lipase, the yields of 4-octyl itaconate were 93%, 60%, 58%, 62%, 50%, 55% and 45%, respectively. It can be seen that the conversion rate of 4-octyl itaconate prepared by the lipases of the present invention are all relatively high. When Novozym 435 was used as catalyst, the conversion rate is more outstanding, due to its higher unit enzyme activity and the shallow active pocket, which facilitates the entry of reaction substrates. The yields of 4-octyl itaconate obtained with other lipases as catalysts are slightly lower due to the deep active pockets, and the entry of reaction substrates is more difficult.

We claim:

1. An enzymatic method for selective catalytic preparation of 4-octyl itaconate, wherein, comprising the following steps: using an itaconic acid and an n-octanol or an n-octanol-derived ester as raw materials to perform an esterification, and using a lipase to catalyze the esterification, wherein a molar ratio of the itaconic acid to the n-octanol or the n-octanol-derived ester is 1: (2-60); after the esterification is completed, conducting extraction to remove itaconic acid, rotary evaporation to remove trace water and solvent, and thermal separation to separate out octanol from 4-octyl itaconate.

2. The enzymatic method for selective catalytic preparation of 4-octyl itaconate according to claim 1, wherein, the lipase is derived from animals, plants and microorganisms.

3. The enzymatic method for selective catalytic preparation of 4-octyl itaconate according to claim 2, wherein, the lipase is selected from the group of Novozym 435, Lipozyme RM IM, Lipozyme TL IM, Yarrowia lipolytica lipase, porcine pancreas lipase, Rhizopus lipase and Carica papaya lipase.

4. The enzymatic method for selective catalytic preparation of 4-octyl itaconate according to claim 1, wherein, an amount of the lipase is 1%-200% of the itaconic acid, based on mass.

5. The enzymatic method for selective catalytic preparation of 4-octyl itaconate according to claim 1, wherein, temperature of the esterification is 5-95° C., and reaction time of the esterification is 4-120 hours.

6. The enzymatic method for selective catalytic preparation of 4-octyl itaconate according to claim 1, wherein, the esterification is carried out in a solvent system or a solvent-free system.

7. The enzymatic method for selective catalytic preparation of 4-octyl itaconate according to claim 6, wherein, the esterification is carried out in a solvent system, and an amount of the solvent is 0.2-10 times of the volume of the n-octanol or the n-octanol-derived ester.

8. The enzymatic method for selective catalytic preparation of 4-octyl itaconate according to claim 1, wherein, the esterification is carried out under a normal pressure or a reduced pressure being 800-1200 pa.

9. The enzymatic method for selective catalytic preparation of 4-octyl itaconate according to claim 1, wherein, after the esterification is completed, conducting extraction to remove itaconic acid, rotary evaporation to remove trace water and solvent to obtain a crude product, then conducting thermal separation to the crude product under the condition of a vacuum degree of 0.5-100 pa and a temperature of 20-100° C. to separate out a white solid, namely the 4-octyl itaconate.

10. The enzymatic method for selective catalytic preparation of 4-octyl itaconate according to claim 9, wherein, the thermal separation is selected from the group of vacuum distillation, rotary evaporation, and short-path distillation.

11. An enzymatic method for selective catalytic preparation of 4-octyl itaconate, wherein, comprising the following steps: using an itaconic acid and an n-octanol or an n-octanol-derived ester as raw materials to perform an esterification, and using an enzyme preparation with a catalytic triplet structure (Ser-His-Asp or Ser-His-Gly) to catalyze the esterification, wherein a molar ratio of the itaconic acid to the n-octanol or the n-octanol-derived ester is 1: (2-60); after the esterification is completed, conducting extraction to remove itaconic acid, rotary evaporation to remove trace water and solvent, and thermal separation to separate out octanol from 4-octyl itaconate.
